(19) United States
(12) Patent Application Publication
(10) Pub. No.: US 2022/0267817 A1
(43) Pub. Date: Aug. 25, 2022

(54) Natural Sweetener Compositions

(71) Applicant: Loren Miles, Encino, CA (US)

(72) Inventors: Loren MILES, Encino, CA (US); Ayesha DOBYNS, Encino, CA (US)

(73) Assignee: Loren Miles, Encino, CA (US)

(21) Appl. No.: 17/180,723

(22) Filed: Feb. 20, 2021

(51) Int. Cl. C12P 19/02; A23L 29/30; A23L 33/125

(52) U.S. Cl. CPC C12P 19/02; A23L 29/30; A23L 33/125; A23L 27/88

(57) ABSTRACT

A natural sweetener composition including a low-calorie monosaccharide sugar (D-allulose), cane sugar and monk fruit extract, to achieve an organoleptic sensation nearly identical to that of cane sugar (sucrose), having an appropriate blend of the D-allulose, the cane sugar and the monk fruit extract, which can result in sweetener product that has many of the desirable properties of sugar without the added calories and glycemic load.

### CROSS-REFERENCE TO RELATED APPLICATIONS

This Application claims priority from U.S. Provisional Application 69/980,007 filed 21 Feb. 2020, which is hereby incorporated by reference.

### FIELD

The present composition relates to a natural sweetener for baking composition. More specifically, the present composition relates to a 50% reduction in calorie natural sweetener composition including a low-calorie monosaccharide sugar (D-allulose), cane sugar and monk fruit extract, and is intended to be used in foods for baking that require the same functionality as sugar and/or food and beverage products to replace and or reduce high-calorie sweetener content such as sucrose.

### BACKGROUND

High-fructose corn syrup, artificial sweeteners, sugar, obesity and diabetes are today's biggest health concerns. Health officials and scientists are beginning to understand in particular the link with high-fructose corn syrup, artificial sweeteners and sugar products to obesity, diabetes and numerous other health concerns. It is estimated that the average American consumes 20 teaspoons a day or more than 150 pounds of sugar per year. Teen consumption is higher at 34 teaspoons of sugar a day. Excessive consumption of sugar, especially by American youth, has experts calling the recent dramatic rise in type 2 diabetes among adolescents an “emerging epidemic.” Further, as a result of excessive refining, sugar is “devoid of vitamins, minerals and fiber.” Accordingly, the statistics are staggering; 129 million adult Americans are overweight, over 60 million, or over 30% of the adult population, are obese. As a result of these health conditions, there are over 300,000 premature deaths each year. Over 40 million children, including “tweens” and teens are overweight. Many health experts assert that sugar is nutritionally empty and detrimental to human health and many believe sugar would fail the FDA approval process if such a hypothetical attempt were made today. According to Dr. Robert C. Atkins, founder of the popular Atkins diet, “sugar has no nutritional value and is directly harmful to your health. Despite vociferous attempts to defend it, there are studies that clearly show how harmful (and even deadly in the case of diabetics) its effects can be.” According to Nancy Appleton, PhD, author of “Lick the Sugar Habit,” there are 78 metabolic consequences of consuming sugar.

Diabetes however, is really only one of the numerous consequences of over-consuming sugar. Dr. John Yudkin, a leading authority on dietary sugars, says that the detrimental effects of excess sugar in the diet go far beyond rotting teeth and obesity. “For example,” Yudkin says, “Sugar causes irregularities in the insulin response; Sugar causes diabetes-like damage to the kidneys; it contributes to degeneration of the retina; it raises blood fat levels and it increases the stickiness of the blood platelets, a common precursor of heart trouble.”

The most effective way to achieve and sustain healthful weight-loss is by reducing calorie intake. Unfortunately, most humans instinctively are attracted to the sensation of sweetness which makes it more difficult for them to resist eating foods and beverages which contain high-caloric, high-glycemic sugars and sweeteners such as, for example, sucrose, fructose, honey and high-fructose corn syrup. Further, food manufacturers that produce low-or reduced-fat products often substantially increase the sugar or sweetener content of products of the food manufacturers to offset the loss of taste and texture often associated with reducing fat content.

One avenue in an attempt to solve these serious health issues is the creation of a 50% reduction in calorie sweetener or sugar substitutes that can be used in foods for baking and/or beverages to replace or reduce high-calorie sweeteners and/or sugar content. Examples of such calorie reducing artificial sweeteners include, for example, aspartame, acesulfame-K, sucralose and saccharin. Low-calorie natural sweeteners would include monk fruit extract (lo han guo) and stevia both derived from fruit and roots, respectively. However, not all zero- or low-calorie sweeteners or sugar substitutes, artificial or natural are suitable for all applications. For example, some sweeteners may be suitable for beverages such as sodas and drink mixes but are not acceptable for use in baked goods because exposure to higher temperatures during baking can reduce the sweetening ability of the sweetener, or lack similar functionality to sugar, such as allowing for the food recipe to rise, caramelize and provide a crust. Some natural sweeteners have a bitter aftertaste and do not render a sweet enough taste or exist in a natural color such as brown or yellow, which conflicts with clear beverages or light colored baked products. As another example, some sweeteners may be suitable for use in solid foods or baked goods but may not work properly for use in beverages and drink mixes due to limitations on solubility or may not have GRAS status (generally recognized as safe as defined by the FDA).

### SUMMARY

In light of the aforementioned, there is a clear need and a demand for a natural sweetener composition for baking food that provides the same functionality as sugar and meets many of the health and commercial requirements such as caloric reduction. Specifically, there is a need and an unmet demand for a 50% reduction in calorie natural sweetener composition that can be utilized in food products such as baked goods, snack foods, dairy products and the like and in beverages and beverage mixes. There is a further need and a demand for a natural sweetener composition for baking which is diabetic safe, tastes like sugar, and provides the same functionality as sugar, such as allowing for the baked food item to rise, caramelize and provide a crust.

Certain embodiments of the present composition include, but are not limited to, a general objective to provide a (100%) natural sweetener composition.

A more specific objective of certain embodiments of the present composition is to provide a natural, 50% reduction in calorie natural sweetener composition that may be used in food products for baking and/or beverages to replace and measures one-to-one volumetrically to sugar in order to easily replace sugar from existing formulas and or recipes.

A further objective of certain embodiments of the present composition is to provide a natural sweetener composition that tastes like sugar, is diabetic safe, and provide functionality similar to sugar, such as allowing for the baked food item to rise, caramelize and provide a crust.

The general object of certain embodiments of the present composition can be attained, at least in part, through a natural sweetener composition including a low-calorie monosaccharide sugar (D-allulose), cane sugar) and monk fruit extract, and to achieve an organoleptic sensation nearly identical to that of cane sugar (sucrose). A sensory sampling was conducted at an international food trade conference with over 200-respondents comprising of professional baker's, chefs, baked food executives and other food industry experts who sampled the composition and having expert sensory skills were unable to distinguish the composition from real sucrose.

In accordance with certain preferred embodiments, the natural sweetener composition includes a low-calorie monosaccharide sugar (D-allulose) derived from fermented corn, cane sugar and monk fruit extract.

In accordance with certain other preferred embodiments, the natural sweetener composition delivers an organoleptic sensation nearly identical to that of cane sugar (sucrose) and includes about 35 to about 75 composition weight percent of D-allulose, 20 to about 60 composition weight percent cane sugar and a 0.1 to about 5 composition weight percent of monk fruit extract.

In accordance with a further preferred embodiment of the composition, the natural sweetener composition includes about 55.5 composition weight percent of D-allulose, 44.4 composition weight percent cane sugar and 0.1 composition weight percent of monk fruit extract.

Previous attempts to solve the solve the above-mentioned problems have generally failed to provide a low-calorie natural baking sweetener composition including a polyol such as erythritol, a reduced-calorie sweetener such as a blend of stevia, monk fruit, oligofructose, fructose, natural flavors, and natural, organic cane sugar (sucrose) that is as effective as desired in satisfying one or more of the above identified needs or demands.

Other objects and advantages will be apparent to those skilled in the art from the following description.

### BRIEF DESCRIPTION OF THE DRAWINGS

FIG. 1 shows an exemplary flowchart illustrating the blending of a natural sweetener, according to one implementation of the present disclosure.

### DESCRIPTION

A certain embodiment of the present composition provides a 50% reduction in calorie natural sweetener composition suitable for use in baked food products and/or food, beverage products, as the composition is designed to achieve an organoleptic sensation nearly identical to that of cane sugar (sucrose). Such a natural sweetener composition includes D-allulose, cane sugar and monk fruit extract. Another certain embodiment of the present composition further provides a natural sweetener composition having a 50% reduction in calories and maintain functionality similar to sugar, such as allowing for the baked food item to rise, caramelize and provide a crust. Yet another certain embodiment of the present composition additionally provides a natural sweetener composition that tastes like sugar, and is diabetic safe.

It is particularly desirable in the production of sweeteners and sugar substitutes to develop sweeteners and sweetener compositions that are as similar as possible to sugar in texture, taste, and usability. More specifically, it is particularly desirable to develop sweeteners and sweetener compositions that provide an appropriate level of sweetness, have similar baking properties to sugar, such allowing a food item to rise, caramelize and provide a crust, have an appropriate texture and mouthfeel, have a positive impact on product appearance, have limited or no negative aftertaste, and provide flavor enhancing and/or masking abilities and stability. However, due to the varying properties of different sweetening ingredients, it is often difficult to achieve as satisfactory of a result when combining such ingredients. This is generally believed to be due to the synergistic effects of mixing various sweetening compounds wherein the sweetness of the mixture is often greater than the apparent sweetness of the individual components. It has now been discovered that an appropriate blend of D-allulose, cane sugar and monk fruit extract can result in a sweetener product that has many of the desirable properties of sugar without its greatest liabilities; a high caloric and glycemic load.

While in the foregoing specification this composition has been described in relation to certain preferred embodiments thereof, and many details have been set forth for purposes of illustration, it will be apparent to those skilled in the art that the natural sweetener composition according to this composition is susceptible to additional embodiments and that certain of the details described herein can be varied significantly without departing from the basic principles of the composition.

Further, in accordance with an embodiment of the present composition, a natural sweetener composition for the primary use in baked food products and/or food, beverage products where the taste and qualities of cane sugar are nearly identical, such as includes D-allulose, cane sugar and monk fruit extract.

In particular, the natural sweetener composition includes: about 25 to about 85 composition weight percent of D-allulose, about 20 to about 70 composition weight percent cane sugar and about 0.01 to about 20 composition weight percent of monk fruit extract.

Preferably, the natural sweetener composition may include additional ingredients, compounds or sweeteners which may enhance the flavor, stability, and processability of the sweetener composition. For example, the natural sweetener composition may include an additional sweetener such as a reduced-calorie sweetener, a sugar-derived sweetener, or a non-nutritive sweetener and or a flavor. Additionally, the natural sweetener composition may include a processing and/or stability aids such as, for example, silica and or other flow or preservative agents.

### EXAMPLE, NATURAL SWEETENER

| | Composition Weight | Single Serving |
|---|---|---|
| Ingredient | Percent | Weight (g) |
| D-Allulose | 55.5% | .555 |
| Cane Sugar | 44.4% | .444 |
| Monk Fruit Extract | .1% | .001 |
| TOTAL | 100% | 1.000 |

While in the foregoing specification this composition has been described in relation to certain preferred embodiments thereof, and many details have been set forth for purposes of illustration, it will be apparent to those skilled in the art that the natural sweetener composition according to this composition is susceptible to additional embodiments and that certain of the details described herein can be varied significantly without departing from the basic principles of the composition.

Further, in accordance with certain preferred embodiments of the present composition, a natural sweetener composition for the primary use in commercial baked food and food, beverage product applications where the taste and qualities of cane sugar are nearly identical, however, to be able to measure one-to-one volumetrically to sugar in order to easily replace sugar from existing formulas and or recipes, offers greater cost efficiency allowing the commercial user or consumer to use their originally formulated recipes, includes an appropriate blend of D-allulose, cane sugar and monk fruit extract, which can result in sweetener product that has many of the desirable properties of sugar without the added calories and glycemic load.

In accordance with certain embodiments of the present composition, a natural sweetener composition for use in beverages and/or food products includes corn derived D-allulose, cane sugar and monk fruit extract. In particular, the natural sweetener composition includes: about 25 to about 85 composition weight percent of D-allulose, about 20 to about 70 composition weight percent cane sugar and about 0.01 to about 20 composition weight percent of monk fruit extract.

Preferably, the natural sweetener composition may include additional ingredients, compounds, flavors or sweeteners which may enhance the flavor, stability, and processability of the sweetener composition. For example, the natural sweetener composition may include an additional sweetener such as a reduced-calorie sweetener, a sugar-derived sweetener, or a non-nutritive sweetener. Additionally, the natural sweetener composition may include a processing and/or stability aids such as, for example, silica and or other flow or preservative agents.

While in the foregoing specification this composition has been described in relation to certain preferred embodiments thereof, and many details have been set forth for purposes of illustration, it will be apparent to those skilled in the art that the natural sweetener composition according to this composition is susceptible to additional embodiments and that certain of the details described herein can be varied significantly without departing from the basic principles of the composition.

### Special Blending Protocol in the Manufacturing Process

To achieve the full benefit of the sweetness level intended and for all of characteristics to be fully realized for these products that the smallest amount ingredient be mixed with the next larger amount until fully blended. Then the next largest ingredient is then added to the combined blend until the next largest ingredient is fully dispersed. Then the next largest ingredient is added after that, and this protocol is continued until all ingredients are blended. This assures maximum dispersion of all elements.

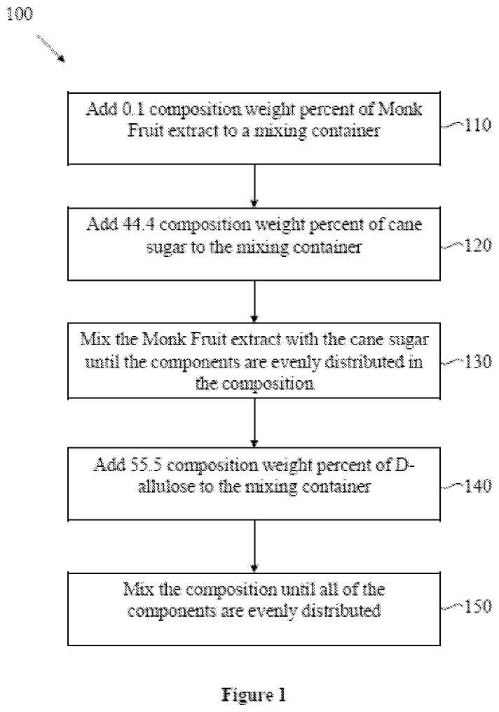

FIG. 1 shows an exemplary flowchart 100 illustrating the blending of a natural sweetener, according to one implementation of the present disclosure. At 110, 0.1 composition weight percent Monk Fruit extract is added to a mixing container, such as a mixing bowl. At 120, 44.4 composition weight percent of cane sugar is added to the mixing container. At 130, the Monk Fruit extract and cane sugar are mixed together until they are evenly distributed in the composition. At 140, 55.5 composition weight percent of D-allulose is added to the mixing container. At 150, the composition is mixed until the Monk Fruit extract, the beet sugar, and the D-allulose are evenly distributed in the composition.

Loren Miles, CEO of Natur Sweeteners, Inc. instructed Ayesha Dobyns, Food Scientist to explore multiple iterations of a natural baking sweetener distributed by Natur Sweeteners, Inc. using a newly developed ingredient known as D-allulose, reduced amounts of sugar and monk fruit extract, believing that the right combination of these three elements could perhaps provide a sensory profile that could mimic the baking properties of sugar to the end user, yet dramatically reduce caloric load. Mr. Miles believed that utilizing reduced amounts of sugar could trick the taste buds in believing it was tasting actual sucrose, while supplanting volume with D-allulose (it has 70% sweetness as compared to sucrose, yet is low-glycemic), and the utilizing trace amounts of potent monk fruit extract to counter balance the sweetness lost using D-allulose.

The amount of use of each ingredient directly influences the sensory experience and caloric load of the formula. For example, too much sucrose defeats the caloric, glycemic load of the formula and intention of the composition. Too little of sucrose defeats the immediate onset response that only sucrose can provide. (Delayed onset of natural sweeteners is an immediate give away that a prompt and radiant mouthfeel that sucrose provides is missing).

Too much D-allulose would require the addition of monk fruit to counterbalance the diminished 70% of sweetness as compared to sugar. Too little of D-allulose and the monk fruit extract begins to have a linger factor that remains in the mouthfeel after the initial burst of sweetness occurs. Sucrose does not have a lingering mouthfeel and the sweetness dissolves very quickly and evenly.

Too much monk fruit extract, conveys an aftertaste effect similar to “Juicy-Fruit” gum and is a dead giveaway of a natural sweetener NOT tasting similar to sucrose. Such an aftertaste also has a tendency to linger, which is unlike a sucrose sensory experience. Too little of monk fruit extract and you have a natural sweetener formula that tastes less than sucrose in sweetness.

Intermixed in this balancing act is the fact that the respective caloric and glycemic loads of the three-ingredients cannot exceed 7.5-calories per gram serving, which the FDA has established as the maximum caloric limit required in order to make a 50% calorie claim. Therefore, even though the sensory experience of the final formula may mimic sucrose identically, if the caloric load associated with each of those three-ingredients exceed the 5-calorie limit, then that formula must be either be modified to meet the legal limit or discarded completely.

Natur Sweetener's Lab Notebook, Titled Natur Sweeteners Lab & Formulary Notes “2019, 2020” respectively, shows notations made by Natur Sweetener's Food Scientist, Ayeesha Dobyns, entered into on the 4th of December, 2019, formula #10 (number of iterations) whereas a formula of the following combined ingredients were sensory tested:

| | | |
|---|---|---|
| A. | D-allulose | 55.5% |
| B. | Cane Sugar | 44.4% |
| C. | Monk Fruit Extract | 0.1 |
| Total | | 100% |

The Lab Notebook will show that Ms. Dobyns and Mr. Miles sampled and tested numerous iterations of this formula commencing on the 1st of August, 2019, and would serve as the initial baseline for the new composition. However, the caloric and glycemic load appeared to exceed the 2-calorie per gram maximum in order to make a 50% less calories than sugar claim established by the FDA and therefore could not be used.

In order to reduce the caloric load, the Food Scientist then made an adjustment to the formula by adjusting the ingredients. Further, numerous iterations of maillard reaction test were taken to determine the right combination of ingredients that would “brown” and provide a “crust” like sugar when the blend was baked and also in sugar cookie recipes.

The Lab Notebook will show that numerous formulas baked “too light” or “too dark” and was rejected. Multiple iterations were made.

On the 1st of August, 2019, Formula F1 tested the Maillard effect.

On the 10 of September, 2019, Formula F3 replaced different ingredients for functionality with no positive result.

On the 30 of September, 2019, Formula F6 found that the omission of ingredient QQ and addition of ingredient X improved caramelization. A determination with the sensory team was that the product was “80% there.”

On the 28th of November, 2019, Formula F9 added an additional ingredient that made the baked product “too soft and not crunchy.”

On the 4th of December, 2019, Formula F10 was determined to be the best and was used to replace sugar in production of over 1,000 brownie samples distributed at the Winter 2020 Fancy Food Show in San Francisco which received overwhelming positive response from recipients who sampled the baked item. Attendees of this even are food retail buyers, professional bakers and other executives in the food and baking industry.

1. A natural sweetener composition including a low-calorie monosaccharide sugar (D-allulose), cane sugar and monk fruit extract, to achieve an organoleptic sensation nearly identical to that of cane sugar (sucrose), having an appropriate blend of the D-allulose, the cane sugar and the monk fruit extract, which can result in sweetener product that has many of the desirable properties of sugar without the added calories and glycemic load.

2. The natural sweetener composition of claim 1 further comprising allowing a food item to rise, caramelize and provide a crust, have an appropriate texture and mouthfeel, have a positive impact on product appearance, have limited or no negative aftertaste, and provide flavor enhancing and/or masking abilities and stability.
